(19) United States
(12) Patent Application Publication
(10) Pub. No.: US 2012/0077876 A1
(43) Pub. Date: Mar. 29, 2012

(54) Method For Improved Bioactivation Of Pharmaceuticals

(75) Inventors: Bernd Clement, Kiel (DE); Dennis Schade, Kiel (DE)

(73) Assignee: Dritte Patentportfolio Beteiligungsgesellschaft Mbh & Co. KG, Schonefeld/Waltersdorf (DE)

(21) Appl. No.: 13/143,866

(22) PCT Filed: Jan. 8, 2010
(86) PCT No.: PCT/DE2010/000009

§ 371 Date: Oct. 3, 2011

(30) Foreign Application Priority Data
Jan. 9, 2009 (DE) 10 2009 004 204.0

(51) Int. Cl. A61K 31/351; C07C 257/18; C07C 279/18; C07C 277/08; A61K 31/196; A61P 33/02; A61P 7/02; A61P 9/00; A61P 25/00; A61P 31/12; A61P 31/16; A61P 31/18; C07D 309/28; A61P 35/00

(52) U.S. Cl. 514/459; 549/424; 562/440; 562/439; 514/566; 514/565; 514/564

(57) ABSTRACT

This invention relates to a prodrug comprising a partial structure having the general formula (I) or (II), where R¹ and R² are hydrogen, alkyl, or aryl radicals.

The present invention relates to a method for improving the bioactivity of pharmaceuticals.

The requirement for a therapeutic effect of a pharmaceutical after oral administration is the absorption thereof from the gastrointestinal tract. The most important mechanism of such an effect is passive diffusion. The degree of resorption by way of passive diffusion is dependent, inter alia, on the lipophilicity.

Another problem with the treatment of many diseases by drugs is the necessity to pass the blood-brain barrier. The blood-brain barrier constitutes an effective barrier with respect to the absorption of substances in the brain. It assures selective take-up and prevents substances from penetrating. Moreover, the blood-brain barrier acts not only as a physical but also as an enzymatic barrier. A variety of processes are involved in the penetration of substances into the brain. In comparison with other indications, only few pharmaceuticals are on the market which manifest the effect thereof in the central nervous system (CNS). Of these, the predominant part reaches the CNS by way of diffusion. In this way, diseases such as epilepsy, chronic pain or depression are treated. Other severe functional disorders such as brain tumors or amyotrophic lateral sclerosis, for example, are very difficult to treat this way today.

So as to be able to overcome biomembranes by way of passive diffusion, a substance should be lipophilic, have a molecular weight lower than 500 Da and it should be present in the uncharged state. To specifically absorb small, highly polar molecules such as amino acids or sugar, different transporter systems such as nucleoside transporters, influx and efflux transports for organic anions or cations, glucose transporters, peptide transporters and amino acid transporters, for example, are expressed at the biomembranes with barrier function (gastrointestinal tract, blood-brain barrier).

For this reason, a variety of prodrug systems are employed to improve the pharmacokinetic properties. A prodrug is a pharmaceutical that is pharmacologically inactive or substantially inactive and is not converted into an active metabolite until it is metabolized in the organism.

N-hydroxyamidines (amidoximes) and N-hydroxyguanidines represent known prodrug principles for increasing the oral bioavailability of amidines [Clement, B. Methoden zur Behandlung und Prophylaxe der Pneumocystis carinii Pneumonic (PCP) und anderen Erkran-kungen sowie Verbindungen und Formulierungen zum Gebrauch bei hesagten Methoden. [DE 4321444] [Methods for the treatment and prophylaxis of Pneumocystis carinii pneumonia (PCP) and other diseases and compounds and formulations for use in said methods] and guanidines. The nitrogen atoms of the amino and imino groups are present in a mesomeric equilibrium in the salts of the amidines and guanidines, and the concepts can he employed for both nitrogen atoms.

The conversion into an active metabolite takes place via different enzyme systems, depending on the underlying prodrug concept. The enzyme system that occurs practically in all forms of live is cytochrome P450 (CYP450), which catalyzes, inter alia, the following reactions:

N-oxidation, S-oxidation, N-dealkylation, O-dealkylation, S-dealkylation, desamination, dehalogenation and hydroxylation of aromatic and aliphatic compounds.

The implication of the diversity of the CYP450 enzyme system is that different substrates and pharmaceuticals compete with the system during the conversion. This results in interactions, reciprocal effects and undesirable mutual influencing. For this reason, CYP450-independent bioactivation is desirable when developing prodrugs.

It is therefore the object of the invention to provide a prodrug system which employs a path of bioactivation that is independent of the cytochrome P450 (CYP450) enzyme. This object is achieved by the subject matter described in the claims. The dependent claims provide advantageous embodiments of the invention.

According to the invention, the object is achieved in one aspect by a prodrug comprising a partial structure having the general formula (I) or (II)

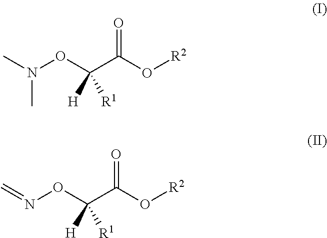

where R¹ and R² are hydrogen, alky radicals or aryl radicals.

In a preferred embodiment of the invention, the term “partial structure”, as it is used herein, denotes that the structural element indicated in the respective formula is part of the formula of substance, preferably of a prodrug. For example, the compound O-carboxymethyl benzamidoxime (1) constitutes a corresponding prodrug of the pharmaceutical benzamidine, wherein the partial structure is a partial structure of formula (II), and R¹ and R² are hydrogen atoms, respectively. This partial structure is a substituent on a benzene ring and together with the same constitutes the pharmaceutical benzamidine.

In a preferred embodiment of the invention, the term “prodrug”, as it is used herein, denotes a substance that as such as inactive or pharmacologically substantially inactive, which is not converted into a pharmaceutical that is pharmacologically active until it is metabolized in the organism. The prodrug can, but does not have to, exhibit improved oral bioavailability than the actual active pharmaceutical. As an alternative, it is possible to use a prodrug because, in comparison with the pharmaceutical, it exhibits improved solubility, bioactivation, blood-brain barrier crossing, physical-chemical stability, lower toxicity and/or a tolerable or more pleasant flavor. For example, erythromycin A 2′-ethyl succinate is not administered as a prodrug to children due to the bitter taste, and not perhaps because of inadequate resorption or solubility of erythromycin A (Bhadra et al. (2005), J. Med. Chem.).

In a further preferred embodiment of the invention, the original prodrug is not metabolized from the prodrug into the pharmaceutical in a one-step reaction, but rather by way of a plurality of reaction steps, wherein each of the metabolites obtained from a reaction step can exhibit one or more of the same and/or different more advantageous properties compared to the original prodrug. To this end, not all of the metabolites may exhibit advantageous properties over the prodrug. For example, a first metabolization product of the prodrug can exhibit increased pharmacological activity compared to the prodrug, a second metabolization product derived from the first metabolization product can likewise exhibit increased pharmacological activity compared to the prodrug, and a third metabolization product derived from the second metabolization product can exhibit increased blood-brain barrier crossing and physical-chemical stability compared to the prodrug.

In a preferred embodiment of the invention, the term “physical-chemical structure”, as it is used herein, denotes the capacity of a substance, for example a prodrug or a pharmaceutical, to be stored and/or used in the form of a relevant aqueous solution, for example dissolved in water, a buffer or a physiological salt solution, without chemical decomposition, for example hydrolysis. In a further preferred embodiment of the invention, the term, as it is used herein, denotes that the substance can be synthesized in stable and synthetic form. In a further preferred embodiment of the invention, the term, as it is used herein, denotes that, during the synthesis of the substance, isolated relevant synthesis precursors are more stable than analogous products, precursors or intermediate products of other substances produced according to an analogous or identical synthesis strategy, so that subsequent synthesis products or synthesis intermediate products can be produced in a more stable form, or can be produced at all.

In one embodiment, the object is achieved by a prodrug, characterized in that the partial structure which the prodrug comprises is part of a hydroxylamine, an N-oxide, a nitron, a diazeniumdiolate (NONOat) or a similar N—O-containing nitric oxide donor, a hydroxamic acid, a hydroxyurea, an oxime, an amidoxime (N-hydroxyamidine), an N-hydroxyamidinohydrazone or an N-hydroxyguanidine.

In the case of the prodrug carboxymethyl benzamidoxime (1) of the pharmaceutical benzamidine, for example, the partial structure is a partial structure of the formula (II) R¹ and R² are hydrogen atoms, respectively, and the partial structure that the prodrug comprises is part of an amidoxime (N-hydroxyamidine).

In one embodiment, the object is achieved by a prodrug, characterized in that the prodrug is metabolized into a pharmaceutical, which is a pharmaceutical for treating diseases associated with nitric oxide deficiency.

In one embodiment, the object is achieved by a prodrug, characterized in that the prodrug or the corresponding pharmaceutical is selected from the group consisting of protease inhibitors, DNA- and RNA-intercalating compounds, inhibitors of viral enzymes, and N-methyl-D-aspartate receptor antagonists.

In a preferred embodiment of the present invention, the term “higher-level partial structure”, as it is used herein, shall be understood such that this higher-level partial structure comprises a partial structure of formula (I) or (II) on the one hand, and is part of the overall structure of the substance in question on the other hand. For example, in the case of the carboxymethyl benzamidoxime (1) prodrug of the pharmaceutical benzamidine (2), the higher-level partial structure, which here is denoted by (Ia), comprises the partial structure of formula (IIa), where R¹ and R² are hydrogen, and the partial structure, which here is denoted by (Ib), is the partial structure of formula (II), where R¹ and R² are likewise hydrogen.

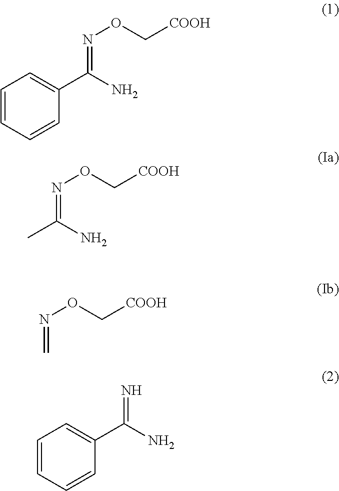

In one embodiment, the object is achieved by a prodrug, characterized in that the partial structure has the general formula IIa or IIb

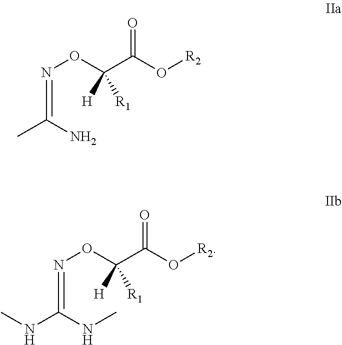

For example, in the case of the carboxymethyl benzamidoximc (1) prodrug of the pharmaceutical benzamidine (2), the higher-level partial structure comprises the partial structure of formula (IIa), where R¹ and R² are hydrogen, the partial structure is the partial structure of formula (II), where R¹ and R² are likewise hydrogen, and the pharmaceutical has the structure (IIa-1) in the prodrug rather than the partial structure of formula (IIa).

In one embodiment, the object is achieved by a prodrug, characterized in that the prodrug is a prodrug of a pharmaceutical, wherein the partial structure of the general formula IIa, after metabolization, comprises a structure having the formula

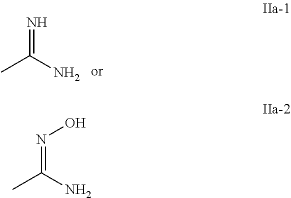

and the partial structure of general formula IIb, after metabolization, comprises a structure having the formula

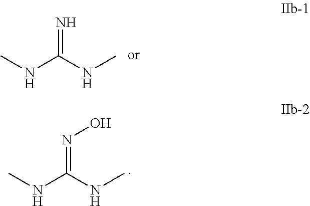

In a further aspect of the invention, the object is achieved by the use of a partial structure forming the general formula (I) or (II)

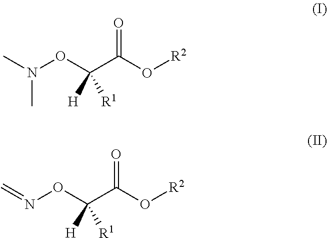

as part of the overall structure of a prodrug which is prodrug or a pharmaceutical, where R¹ and R² are hydrogen, alkyl radicals or aryl radicals.

In one embodiment, the object is achieved by the use of a prodrug, wherein the partial structure has the general formula (II), and is part of a higher-level partial structure IIa or IIb

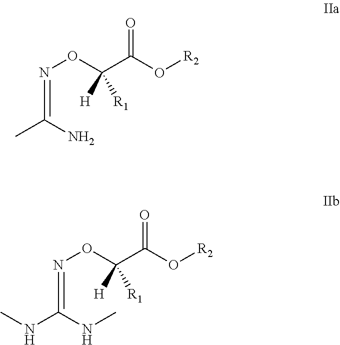

in the place of an amidine or guanidine group of a pharmaceutical to improve solubility, oral bioavailability, blood-brain barrier crossing, the flavor and/or the physical-chemical stability.

In one embodiment, the object is achieved by the use of a prodrug, wherein the prodrug is a prodrug of a pharmaceutical that has the same structure as the prodrug, except that instead of the higher-level partial structure IIa it comprises one of the partial structures IIa-1 or IIa-2

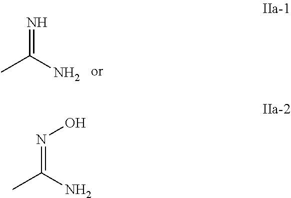

or instead of the higher-level partial structure IIb it comprises one of the partial structures IIb-1 or IIb-2

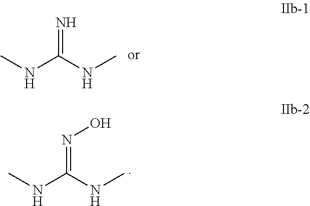

In one embodiment, the object is achieved by the use of a prodrug activating the pharmaceutical by peptidylglycine α-amidating monooxygenase (PAM).

In a preferred embodiment of the invention, the expression “activating the pharmaceutical by peptidylglycine α-amidating monooxygenase (PAM)”, “activating a prodrug by way of the PAM activation path”, bioactivation or the like, as it is used herein, denotes that the prodrug is recognized by PAM as a substrate and metabolized. In a preferred embodiment of the invention, the expression “introducing a pharmaceutical into the PAM activation path, comprising the production of a prodrug of the pharmaceutical”, as it is used herein, denotes that a corresponding prodrug form is produced of a pharmaceutical to be introduced into the PAM activation path, this prodrug form being recognized by PAM and metabolized. In a preferred embodiment, the affinity of the prodrug for PAM, as compared with the pharmaceutical, is 1-1000 times, 2-100 times, 3-50 times, 4-40 times, 5-20 times or even 6-15 times greater, as a person skilled in the art will be able to determine using the K_{M} values.

In one embodiment, the object is achieved by the use of a prodrug, characterized in that the partial structure is part of a hydroxylamine, an N-oxide, a nitron, a diazeniumdiolate (NONOat) or a similar N—O-containing nitric oxide donor, a hydroxamic acid, a hydroxyurea, an oxime, an amidoxime (N-hydroxyamidine), an N-hydroxyamidinohydrazone or an N-hydroxyguanidine.

In a further aspect of the invention, the object is achieved by a method for introducing a pharmaceutical comprising a free amidine or guanidine function into the PAM activation path, comprising the production of a prodrug of the pharmaceutical.

In a further aspect of the invention, the object is achieved by a method for treating a patient, comprising the administration of a prodrug to the patient.

In a further aspect of the invention, the object is achieved by the use of a prodrug for producing a pharmaceutical.

In a preferred embodiment of the invention, the pharmaceutical is a pharmaceutical, or the prodrug is a prodrug, for combating viral infections such as influenza, for combating HIV infections, for the prophylaxis and treatment of visceral and cutaneous leishmaniasis, for the prophylaxis of Pneumocystis carinii pneumonia (PCP), for treating trypanosomiasis (African sleeping sickness), for treating malaria, for treating babesiosis, for inhibiting blood coagulation, for example for the primary prevention of venous thromboembolic events, for the prophylaxis of stroke in patients with atrial fibrillation, for lowering blood pressure, for inhibiting the growth of malignant tumors, for neuroprotection, for combating viral infections such as influenza, for the (diuretic) elimination of water from the body, for example with cardiac insufficiency, pulmonary edema, poisoning, renal insufficiency or cirrhosis of the liver, for treating allergies, for treating asthma, for treating inflammatory diseases, for example rheumatism or pancreatitis, or for the prophylaxis of ischemia (insufficient blood supply).

In a further aspect of the invention, the object is achieved by the use of a prodrug according to any one of claims 7 to 11 and claim 14, or by a method according to claim 13, wherein the use or the method is a use or a method for treating diseases associated with nitric oxide deficiency.

In one embodiment, the object is achieved by the use of a prodrug, characterized in that the pharmaceutical or the prodrug is selected from the group consisting of protease inhibitors, DNA- and RNA-intercalating compounds, inhibitors of viral enzymes, and N-methyl-D-aspartate receptor antagonists.

In one embodiment, the object is achieved by the use of a prodrug, wherein the use is a use for the prophylaxis and/or treatment of visceral and/or cutaneous leishmaniasis, trypanosomiasis, phase 2 of trypanosomiasis or pneumonia caused by Pneumocystis carinii, for inhibiting the growth of malignant tumors, for inhibiting blood coagulation, for lowering blood pressure, for neuroprotection, or for combating viral infections, including influenza and HIV infections.

In a further aspect of the invention, the object s achieved by a pharmaceutical comprising a partial structure having the general formula (I) or (II)

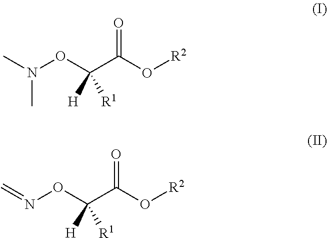

where R¹ and R² are hydrogen, alky radicals or aryl radicals.

In one embodiment, the object is achieved by a pharmaceutical comprising a partial structure having the general formula (I) or (II), characterized in that the partial structure is part of a hydroxylamine, an N-oxide, a nitron, a diazeniumdiolate (NONOat) or a similar N—O-containing nitric oxide donor, a hydroxamic acid, a hydroxyurea, an oxime, an amidoxime (N-hydroxyamidine), an N-hydroxyamidinohydrazone or an N-hydroxyguanidine,

In one embodiment, the object is achieved by a pharmaceutical according to any one of the preceding claims, characterized in that the pharmaceutical is designed to treat diseases associated with nitric oxide deficiency.

In one embodiment, the object is achieved by a pharmaceutical, characterized in that the pharmaceutical is selected from the group consisting of protease inhibitors, DNA- and RNA-intercalating compounds, inhibitors of viral enzymes, and N-methyl-D-aspartate receptor antagonists.

In a further aspect of the invention, the object is achieved by the use of an O-carhoxyalkylated N—O-containing functionality for producing a pharmaceutical comprising a partial structure forming the general formula (I) or (II)

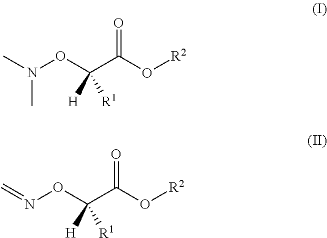

where R¹ and R² are hydrogen, alky radicals or aryl radicals, for improving the solubility, bioavailability, blood-brain barrier crossing, bioactivation and/or the physical-chemical stability of the pharmaceutical.

In one embodiment, the object is achieved by the use of a pharmaceutical comprising an O-carboxyalkylated N—O-containing functionality for activating the pharmaceutical by peptidylglycine α-amidating monooxygenase (PAM).

In one embodiment, the object is achieved by the use of a pharmaceutical, characterized in that the partial structure is part of a hydroxylamine, an N-oxide, a nitron, a diazeniumdiolate (NONOat) or a similar N—O-containing nitric oxide donor, a hydroxamic acid, a hydroxyurea, an oxime, an amidoxime (N-hydroxyamidine), an N-hydroxyamidinohydrazone or an N-hydroxyguanidine.

In one embodiment, the object is achieved by the use of a pharmaceutical, characterized in that the pharmaceutical is designed to treat diseases associated with nitric oxide deficiency.

In one embodiment, the object is achieved by the use of a pharmaceutical, characterized in that the pharmaceutical is selected from the group consisting of protease inhibitors, DNA- and RNA-intercalating compounds, inhibitors of viral enzymes, and N-methyl-D-aspartate receptor antagonists.

In one embodiment, the object is achieved by the use of a pharmaceutical, characterized in that the pharmaceutical is designed for the prophylaxis and/or treatment of visceral and/or cutaneous leishmaniasis, trypanosomiasis, phase 2 of trypanosomiasis or pneumonia caused by Pneumocystis carinii, to inhibit the growth of malignant tumors, to inhibit blood coagulation, to lower blood pressure, for neuroprotection, or to combat viral infections, including influenza and HIV infections.

In a further aspect of the invention, pharmaceutical compounds, pharmaceutical compositions and pharmaceutical products are provided, which comprise the compounds according to the invention and/or the salts thereof. The pharmaceutical compositions preferably contain carriers and/or adjuvants and ideally they are pharmaceutically compatible. A person skilled in the art is generally familiar with such carriers and adjuvants. The compounds according to the invention are also provided for use in medicine.

It is sufficient if the pharmaceutical comprises at least one or more active amidine, N-hydroxyamidine (amidoxime), guanidine or N-hydroxyguanidine functions in the proposed form. The pharmaceutical can thus contain, for example, a plurality of amidoxime functions (for example two, as with pentoxime ester) or N-hydroxyguanidine functions, wherein then at least one of these groups is modified in the aforementioned manner. Similarly, mixtures of pharmaceuticals can also be employed, of which at least one is modified according to the invention.

The compounds according to the invention can he administered once, as a bolus administration, every day, weekly or monthly. The manner of the administration can likewise he easily determined. In general, the possible forms of administration include oral, rectal, parenteral such as intravenous, intramuscular, subcutaneous, transdermal administration, intrapulmonary administration and administration as an aerosol, intravesical instillation, intraperitoneal or intracardiac injection, uptake via mucous membranes or intravaginal application, for example by means of suppositories. The oral form of administration can be a liquid, semi-solid or solid formulation, in particular in the form of tablet, sugar-coated tablet, pellet or microcapsule. To this end, the active ingredient, or the active ingredient mixture, is received in a suitable non-toxic solvent, such as water, monohydric alcohols, in particular ethanols, multihydric alcohols, in particular glycerin and/or propanediol, polyglycols, in particular polyethylene glycols, and/or Miglyol, glycerinformal, dimethyl isosorbide, natural or synthetic oils, for those embodiments in which liquid formulations are used. The conventional base products, such as bentonite, Veegum, guar meal and/or cellulose derivatives, in particular methyl cellulose and/or caboxymethyl cellulose, and polymers made of vinyl alcohols and/or vinyl pyrrolidones, alginates, pectins, polyacrylate, solid and/or liquid polyethylene glycols, paraffins, fatty alcohols, vaseline and/or waxes, fatty acids and/or fatty acid esters are used to produce semi-solid or solid preparations.

Moreover, the known extenders, such as colloidal silicic acid, talcum, lactose, starch powder, sugar, gelatin, metal oxides and/or metal salts may be present in solid formulations. Further additives such as stabilizers, emulsifiers, dispersing agents and preservatives are an obvious choice.

Surprisingly, it has been found that O-carboxyalkylated N—O-containing functionalities of the general formula (I) or (II), which are bound to a pharmaceutical molecule via bonds at the nitrogen (N),

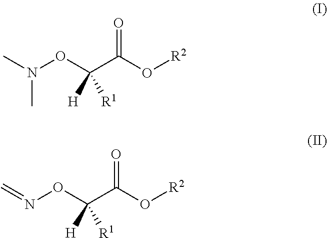

where (I) and (II) are, for example, part of a hydroxyl amine, an N-oxide, a nitron, a diazeniumdiolate (NONOat) or similar N—O-containing nitric acid donor, a hydroxamic acid, an oxime, an amidoxime (N-hydroxyamidine), an N-hydroxyamidinohydrazone or an N-hydroxyguanidine, and R¹ (which must be pro-R configured) and R² are hydrogen, alkyl radicals or aryl radicals, utilize a bioactivation path that is independent of cytochrome P450 (CYP450) enzymes. This constitutes an unexpected result because it is known that CYP450 enzymes generally catalyze oxidative O-dealkylations, which in the case of the prodrug principle proposed here would also be necessary to release the actual pharmaceutical.

The proposed etherification of N—O-containing functionalities with carhoxyalkyl radicals produces the special advantage that an enzyme different from the CYP450 enzyme can be utilized for bioactivation: peptidylglycine α-amidating monooxygenase (PAM). This prevents, for example, side effects and the aforementioned interactions with other simultaneously administered pharmaceuticals.

In higher organisms (vertebrates), peptidylglycine α-amidating monooxygenase (PAM) constitutes a bifunctional enzyme, which is composed of a monooxygenase domain (PHM, peptidylglycine α-hydroxylating monooxygenase, EC 1.14.17.3) and a lyase domain (PAL, peptidyl-α-hydroxyglycine α-amidating lyase, EC 4.3.2.5). On an overall basis, PAM is subject to a strongly tissue-specific and development-dependent regulation by splicing and expression. Within the meaning of a post-translational modification, PAM is able to activate diverse physiologically occurring peptide hormones, neurotransmitters and growth factors (for example, substance P, neuropeptide Y, oxytocin, vasopressin, calcitonin). In the process, the peptides are C-terminally amidated by separating a terminal glycine by means of oxidative N-dealkylation in a monooxygenase reaction.

A particular advantage of the etherification of the N—O-containing functionalities with carboxyalkyl radicals, as proposed according to the invention, is the improved solubility resulting from the insertion of a carboxylic acid that is negatively charged under physiological conditions (pH 6-8).

An additional advantage is that the etherification of the N—O-containing functionalities proposed according to the invention—using (alkoxycarbonyl)alkyl ethers or (aryloxycarbonyl)alkyl ethers—increases the lipophilicity so much that passive diffusion is made possible, whereby the bioavailability and/or blood-brain barrier crossing is improved.

The possibility of using a comparatively small radical—in the simplest case, a carboxymethyl radical—as the prodrug group, so that the size of the pharmaceutical molecule increases only moderately, is likewise advantageous.

Wand et al. [Metabolism 1985, 34, 11, 1044] analyzed PAM activities in different human tissues and detected the highest activity in tissues of the CNS (in particular in the pituitary gland). In contrast, no activity was found in the classic foreign matter-metabolizing organs, the liver and the kidneys. Activities for which the planned prodrug concept could also be utilized were likewise detected in plasma, the heart and lungs.

In particular the high activities of this enzyme in the CNS can be utilized to transport O-carboxyalkylated prodrugs through the blood-brain barrier, so as to then convert them. However, bioactivation in the cardiovascular system after peroral application and absorption from the gastrointestinal tract is also possible.

The prodrug system according to the invention can be applied to different pharmaceuticals which have an amidine or guanidine function. The following pharmaceuticals are particularly preferred:

pentamidine, dabigatran, BSF 411693 (Abbott), idazoxan hydrochloride, irbesartan, linogliride, lofexidine hydrochloride, tetrahydrozoline hydrochloride, tolazoline, xylometazoline hydrochloride, pentamidine isethionate, taribavirin, thiamine (Vitamin B1), bosentan, dibromopropamidine isethionate, hydroxystilbamidinc isethionate, sibrafiban, orboliban, xemilofiban, argatroban, ximclagatran, melagatran, 2-piperidinic acid, orbofiban acetate, epinastine (Relestat), RO 43-8857, AB1 (Chlorambucil, analogues), AMG-126737, AY-0068, B-623, BABIM, BIBT-986 (Boehringer Ingelheim), CI-1031 (company: Biosciences), CJ-1332 (company: Curacyte), CJ-463 (company: Curacyte), CJ-672 (company: Curacyte), CT50728 (Portolla Pharmaceuticals), CVS-3983, DX-9065a, Lamifiban (Roche), LB-30870 (company: LG LifeSciences Ltd), LY-178550 (company: Lilly), PHA-927F and analogues, RO-44-3888 (Roche), sepimostat, FUT-187 (Torii), viramidine (Ribapharm), WX-FX4 (Wilex), YM-60828 (Yamanouchi Pharmaceutical Co. Ltd), ZK-807191 (Berlex Bioseiences), NAPAP (SR 25477), BIIL 315 (Boehringer Ingelheim), BIIL 260 (Boehringer Ingelheim), BIIL 284/260 (Boehringer Ingelheim), tanogitran, moxilubant, stilbamidine, panamidine, fradafiban, diminazene, roxifiban, furamidine, PD0313052, PHA 927F, PHA 798, fidexaban, otamixaban, thromstop (Thrombstop), zanamivir, amiloride hydrochloride, anagrelide hydrochloride, proguanil, cimetidine, clonidine hydrochloride, guanoxan, peramivir, romifidine, tirapazamine, tizanidine, tolonidine nitrate, metformin, diminazene, debrisoquine, sulfamethazine, eptifibatide, famotidine, Bayer pharmaceutical, streptomycin, nafamostat, FUT-175, inogatran, guanethidine (Thilodigon), 3DP-10017, APC-366, CVS-1123, diphenyl phosphonate derivative, E-64, FOY-305, MBGB, MIBG, RWJ-422521, Synthalin, WX-293, WX-340, BMS-189090, JTV-803 (Japan Tabacco), napsagatran, ismelin, Tan 1057A, Hydikal, Phenformix (Retardo), netropsin (Sinanomycin), BIIB 722 (sabiporide), guanadrel, deoxyspergualin, BMS 262084, Siamformet (Orabet), PPACK (Pebac), MERGETPA (Plummer's carboxypeptidase inhibitor), peramivir, famotidine, zaltidine.

The annex contains a table with the chemical formulas, the CAS registry numbers and the indications of the pharmaceuticals.

Hereinafter, 4 prodrugs according to the invention are shown by way of example:

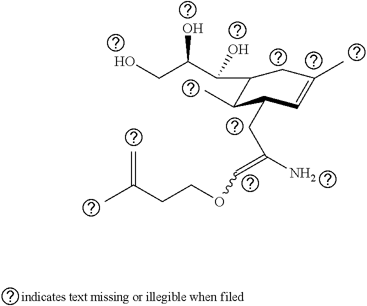

Carboxyethoxy prodrug of zanamivir

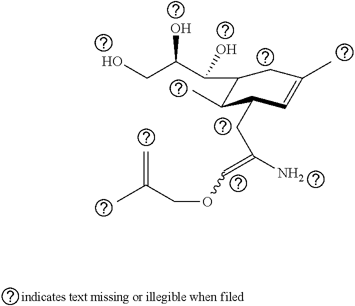

Carboxymethoxy prodrug of zanamivir

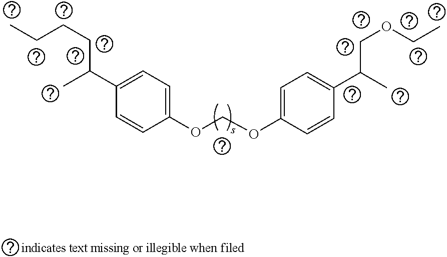

Bis(carboxymethoxy) prodrug of pentamidine

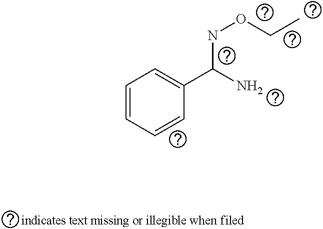

Carboxymethyl benzamidoxime

The surprising discovery that non-peptidic O-carboxyalkylated N—O-containing functionalities are accepted as substrates of PAM is demonstrated in the exemplary embodiments based on amidoxime- and N-hydroxyguanidine-based model compounds.

O-carboxymethyl benzamidoxime (1) was tested for the PAM substrate properties thereof as a model compound of amidoximes. O-carboxymethyl benzamidoxime is a possible prodrug of the pharmaceutical benzamidine. The PAM-catalyzed bioactivation of O-carboxymethyl benzamidoxime (1) into benzamidoxime (2) occurs with glyoxalic acid being released at the same time.

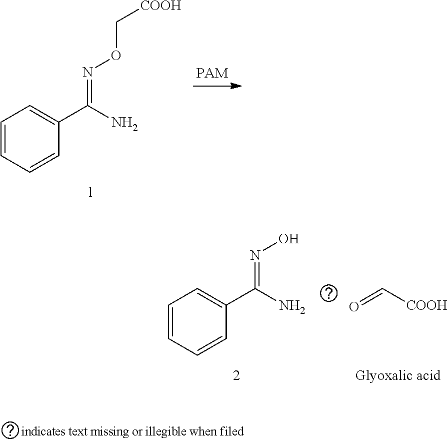

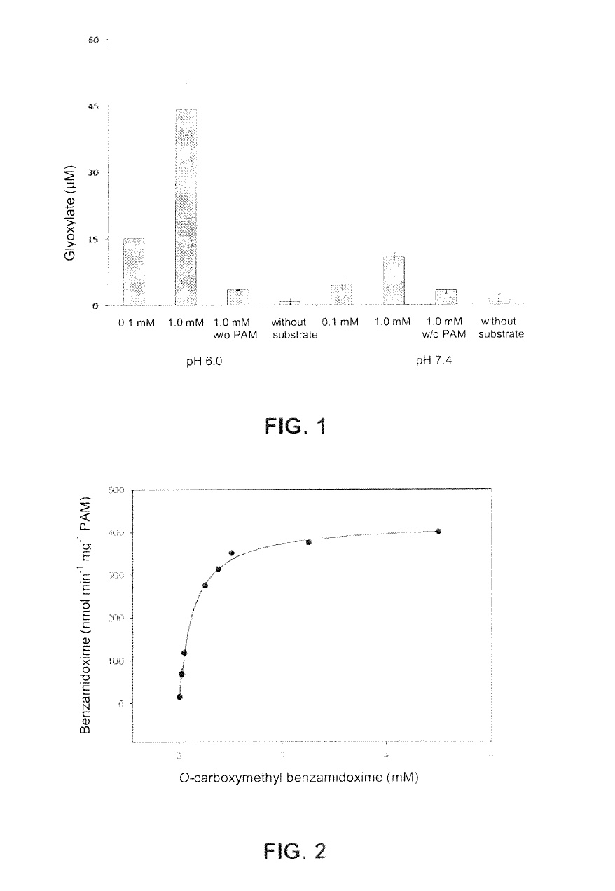

FIG. 1 shows the results of the colorimetric determination of the glyoxalate formation. The determined glyoxylate concentrations are mean values ± standard deviations from two incubations, each of which was measured twice. The formation of glyoxylate as the cleavage product of the PAM catalysis of 1 was verified in an concentration-dependent manner. The incubations at the pH optimum of PAM (pH 6.0) resulted in considerably higher conversions in comparison with the incubation at pH 7.4. In the colorimetric assay, a 5-point calibration of glyoxylate was carried out simultaneously with the testing of 1. The calibration was linear in the determined concentration range (r²=1.000).

Since, according to these results, O-carboxy ethyl benzamidoxime (1) was accepted as a substrate by PAM, the reaction was characterized in more detail by determining the K_{M} and Vₘₐₓ values.

For this purpose, an HPLC analysis was developed. The calibration line for benzamidoxime was linear in the determined concentration range (r²=1.000) and the recovery rate was 130.6% (r²=0.999). Two independent experiments (n=2) yielded a K_{M} value of 307±80 μM and Vₘₐₓ value of 393±40 nmol min⁻¹ mg⁻¹ PAM. FIG. 2 is a representative illustration of such a determination.

For the CYP450 substrate studies, the aforementioned HPLC analysis was modified so that additionally the detection of the conceivable metabolite benzamidine is possible as a product from the N-reduction of benzamidoxime (2). At pH 6.0 and pH 7.4, neither benzamidoxime possible prodrug of benzamidine) nor benzamidine were detected in any of the CYP450 enzyme sources.

Based on the benzamidoxime model compound 1, the O-carboxymethyl function is removed only from PAM, but not from cytochrome P450 within the meaning of a monooxygenase reaction.

N-carboxymethoxy-N′,N″-diphenyl guanidine (3) was tested for the PAM substrate properties thereof as a model compound of hydroxyguanidines.

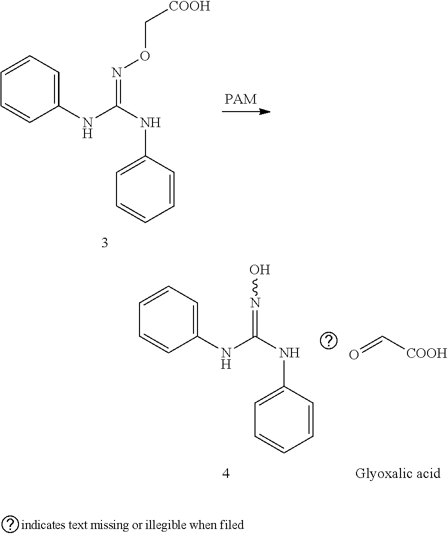

The PAM-catalyzed bioaetivation of N-carboxymethoxy-N′,N″-diphenyl guanidine (3) into N,N″-diphenyl-N″-hydroxy guanidine (4) takes place with glyoxalic acid being released at the same time.

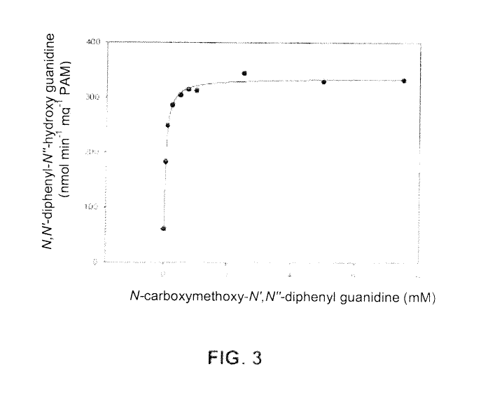

The results from the colorimetric assay using 3 were comparable to those of the amidoxime model compound 1. For determining the K_{M} and Vₘₐₓ values, an HPLC analysis was developed which is able to separate the prodrug 3 and hydroxy guanidine 4 within 15 minutes on an RP column. The calibration line for N,N′-diphenyl-N″-hydroxy guanidine (4) was linear in the determined concentration range (r²=0.999) and the recovery rate was 111.7% (r²=0.999). Two independent experiments (n=2) yielded a K_{M} value of 37±5 μM and Vₘₐₓ value of 373±53 μmol min⁻¹ mg⁻¹ PAM. FIG. 3 is a representative illustration of such a determination.

From the determined K_{M} value, an affinity for PAM that is approximately 8 times greater in comparison with the amidoxime prodrug 1 can be derived, while the conversion rate is comparable.

For the CYP450 substrate studies, the HPLC analysis developed for the PAM substrate studies was modified so that additionally the detection of the conceivable metabolite N,N′-diphenyl guanidine is possible as a product from the N-reduction of hydroxy guanidine 4. At pH 6.0 and pH 7.4, neither 4 nor N,N′-diphenyl guanidine were detected in any of the CYP450 enzyme sources that were used after an incubation time of 180 minutes.

Analogously to O-carboxymethyl benzamidoximc (1), the O-carboxymethyl function is removed only from PAM, but not from cytochrome P450 within the meaning of a monooxygenase reaction, based on the hydroxyguanidine model compound 3.

### MATERIALS AND METHODS

Sodium Salt of O-carboxymethyl benzamidoxime monohydrate (1)

Modified instruction according to Koch [Ber. Dtsch. Chem. Ges. 1889, 22, 3161]:

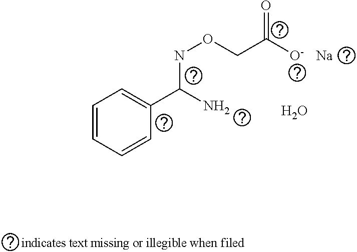

A solution of 681 mg benzamidoxime (5.0 mmol), 1.04 g bromoacetic acid (7.5 mmol) and 500 mg sodium hydroxide pellets (12.5 mmol) in 5 ml ethanol is boiled for 5 hours under reflux. Thereafter, the solvent is removed under vacuum until a deposit starts to form. The deposit is allowed to fully precipitate, filtered off and dried. The product is recrystallized from ethanol (96%)/water (95:5).

Yield: 937 mg white fine felt-like crystals (80%)

Melting pt.: 226° C. (dec.)

1H-NMR (DMSO-d₆):

δ/ppm=4.13 (s, 2H, O—CH₂), 6.09 (br s, 2H, NH₂), 7.37 (m, 3H, 3′,4′,5′-CH), 7.67 (m, 2H, 2′,6′-CH).

13C-NMR (CDCl₃):

δ/ppm=73.6 (O—CH₂), 125.7, 128.0, 129.0 (ArCH), 132.8 (ArC), 151.4 (C═N), 173.2 (CO).

MS (ESI):

m/z=217 [M+Na]⁺, 195 [M+H]⁺, 119 [M-C₄H₂-C₂H₂+H]⁺, 105 [C₆H₅N₂]⁺.

C₉H₉N₂NaO₃.1.0 H₂O (234.18)

Calculated C 46.16 H 4.73 N 11.96

Found C 46.43 H 4.44 N 11.65

N-carboxymetboxy-N′,N″-diphenyl guanidine (3)

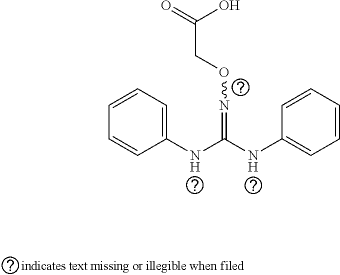

546 mg aminooxyacetic acid semichloride (5 mmol) and 697 μl triethylamine (5 mmol) are stirred for 30 minutes in 10 ml dry DMF. The precipitate is filtered off and 970 mg N,N′-diphenyl carbodiimide (5 mmol) is added to the Filtrate. The batch is stirred for four hours at room temperature, solvent-extracted with ethyl acetate, and the product is recrystallized from ethanol.

Yield: 285 mg of a white solid (20%)

Melting pt.: 176° C.

DC: R_{f}=0.29 (dichloromethane/methanol, 9:1)

1H-NMR (DMSO-d₆):

δ/ppm=4.37 (s, 2H, O—CH), 6.75-6.87 (m, 2H, ArH), 7.03-7.20 (m, 8H, ArH), 8.02, 8.21 (2x br s, 1H, NH), 12.05 (br s, 1H, COOH).

13C-NMR (DMSO-d₆):

δ/ppm=70.0 (O—CH₂), 116.7, 118.7, 119.8 121.0, 128.5 (ArCH), 140.7 (ArC), 142.3 (ArC), 147.5 (C═N), 171.8 (CO).

MS (ESI):

m/z=308 [M+Na]⁺, 286 [M+H]⁺, 210 [M-C₂H₄O₃]⁺.

MS (EI):

m/z (%)=209 (38), 208 (37), 119 (20), 118 (38), 93 (100), 91 (47), 77 (43), 66 (31), 51 (30).

C₁₅H₁₅N₃O₃ 0.3 H₂O (290.71)

Calculated C 61.97 H 5.41 N 14.45

Found C 62.18 H 5.72 N 14.57

### HPLC System

Waters Breeze HPLC system with Waters 1525 pumps, Waters 2487 absorption detector, Waters 717 Plus autosampler and Breeze recording and evaluation software (Version 3.30), Gynkotek STH 585 column oven.

### HPLC Columns:

Synergi Max-RP 80 (250×4.6 mm, 4 μm) with C-18 precolumn (4×3 mm) (Phenomenex);

LiChroCART, LiChrospher 100, RP-8 (125×4 mm, 5 μm) with LiChrospher 60 precolumn, RP-select B (4×4 mm, 5 μm) (Merck);

LiChroCART, LiChrospher RP-select B (250×4.6 mm, 5 μm) with LiChrospher 60 precolumn, RP-select B (4×4 mm, 5 μm) (Merck).

### Additional Devices and Materials:

Cary 50 UV-Vis photometer (Varian); 96-well plates (Greiner); GFL-1083 shaking water bath (Gesellschaft für Labortechnik, Burgwedel); microliter centrifuge (Hettich GmbH); InoLab pH Level 1 pH measuring device (Wissenschaftlich-Technisehe Werkstätten GmbH, Weilheim) with a LiQ Plast pH electrode (Hamilton); VF2 vortexer (Janke und Kunkel GmbH & Co. KG, Staufen); 1.5 ml reaction vessels (Sarstedt AG & Co., Nümbrecht).

### Enzyme Sources:

The recombinant peptidylglycine α-amidating monooxygenase (PAM, rat, EC 1.14.17.3) that was used was provided by Unigene Laboratories, Inc. (New jersey, USA) (specific activity=5.8 10⁶ U/mg protein); bovine liver catalase (EC 1.11.1.6), specific activity=12600 U/mg solid (Aldrich).

The cytochrome P450 enzyme sources that were used were obtained in the Clement von Grünewald working group according to the following instructions:

### Porcine Liver Microsomes and 9000 g Supernatant:

The pork livers were procured from a local butcher (Bordesholm) and the organs were transported directly after slaughter in an ice-cooled 20 mM phosphate buffer (1 mM Na₂ EDTA, pH 7.4). For further processing, the liver lobes were first perfused with 50 mM phosphate buffer (1 mM Na₂ EDTA, pH 7.4) and washed. The tissue was cut into pieces and run through a commercially available meat grinder. The suspension was diluted an equal volume of phosphate buffer and homogenized using a flow homogenizer. The microsomes and 9000 g supernatant were further obtained by differential ultracentrifugation. For storage, the resulting preparations were aliquotted and frozen at −80° C.

### Human Liver Microsomes and 9000 g Supernatant:

To obtain human microsomes, human liver tissue from cancer patients of the surgical department of the University Clinic of Christian-Albrecht University was obtained who had to undergo hemihepatectomy.

The liver tissue pieces were flash-frozen in a saccharose-containing phosphate buffer (10 mM K2HPO4, 10 mM KH2PO4, 250 mM saccharose, 1 mM Na2 EDTA, pH 7.4, 4° C.). As soon as a sufficient quantity of organ parts (>3) was available, the corresponding pieces were thawed and pooled so as to compensate for differences due to interindividual variations. The tissue pieces were cut into smaller parts at 4° C., washed several times with buffer solution (without EDTA), and processed into a suspension using a homogenizer. The microsomes and 9000 g supernatant were further obtained from this suspension by differential ultracentrifugation. For storage, the resulting preparations were aliquotted and frozen at −80° C.

### PAM Assay: Incubation Conditions

A typical incubation batch of 300 μl (total volume) contained 25000 U/ml peptidylglycine α-amidating monooxygenase (PAM, company: Unigene Laboratories), 250 U/ml catalase, 1 μM copper(II) (employed as acetate/monohydrate), 2 mM sodium ascorbate, 5 mM potassium iodide and the respective substrate in 0.1 mM or 1 mM concentration, in buffers having different pH values. The buffer system used was 30 mM MES for the incubation at pH 6.0 and 50 mM HEPES for the incubation at pH 7.4. The pH value was adjusted in each case with diluted sodium hydroxide. The incubation was carried out at 37° C. in the shaking water bath for 60 minutes, 100 μl was withdrawn, and the reaction was stopped with 50 μl 10% TFA_{(aq)/}acetonitrile (2:3). The remaining batch was incubated for another 180 minutes at 37° C. and stopped with 100 μl 10% TFA_{(aq)/}acetonitrile (2:3).

The stopped samples were shaken for 5 minutes (vortexer) and frozen at −80° C. To analyze the samples, they were thawed, shaken for 5 minutes, and the precipitated protein was centrifuged at 10000 rpm. The supernatant was used for the colorimetric glyoxylate determination and/or HPLC measurement.

For the K_{M} and Vₘₐₓ determination, 100 μl batches were treated at pH 6.0 under the aforementioned conditions, however with the difference that the incubation time was 30 minutes.

### Colorimetric Determination of Glyoxylate

200 μl of the incubation batch that was freed of protein was mixed with 20 μl of a phenylhydrazine solution (20 mg in 2 ml aqua bidest.) and shaken for 5 minutes in the shaking water bath at 37° C. Thereafter, the mixture was cooled for 15 minutes to 0° C., 100 μl ice-cold 6 N HCl was added and allowed to sit at 0° C. for an additional 5 minutes. Then, 20 μl of a potassium hexacyanoferrate(III) solution (100 mg in 2 ml aqua bidest.) was added. The batch was allowed to rest for 15 minutes at room temperature and 200 μl was withdrawn for the measurement using a Plate Reader (Cary 50 UV-Vis photometer, 520 nm).

### Calibration:

For a 5-point calibration, glyoxalic acid in concentrations of 2, 5, 10, 50 and 100 μM in a 2:1 mixture of assay buffer (pH 6.0):10% TFA_{(aq)/}acetonitrile (2:3) was measured as described above. This calibration took place simultaneously for each assay of a test compound that was carried.

HPLC Analysis for Separating O-carboxymethyl benzamidoxime (1) and benzamidoxime (2)

Column: Synergi Max-RP 80 A (250×4.6 mm, 4 μm)

Column temperature: 20° C.

Mobile phase: 79% (v/v) 10 mM octyl sulfonate, pH 2.5 (H₃PO₄) 21% (v/v) acetonitrile

Flow rate: 1.0 ml/min

Run time: 20 min.

Detection: Absorption measurement at 229 nm

Injection volume: 20 μL

Retention times:

| | |
|---|---|
| O-carboxymethyl benzamidoxime (1) | 8.9 min + 0.2 min |
| Benzamidoxime (2) | 14.4 min + 0.2 min |

### Calibration and Recovery

For the calibration, benzamidoxime was dissolved in eight concentrations of 0.1-500 μM, dissolved in assay buffer (30 mM MES, 1 μM copper(II) acetate, 2 mM sodium ascorbate, 5 mM potassium iodide, pH 6.0), and measured using the aforementioned HPLC method. For determining the recovery, the same concentrations were produced in assay buffer (end volume=100 μl). In addition, O-carboxymethyl benzamidoxime (0.5 mM) and 250 U/ml catalase were added, followed by 50 μl 10% TFA_{(aq)/}acetonitrile (2:3). The samples were shaken using the vortexer and frozen at −80° C. To measure the samples, they were thawed, shaken 5 minutes using the vortexer, and centrifuged for 5 minutes at 10000 rpm.

HPLC Analysis for Separating N-carboxymethoxy-N′,N″-diphenyl guanidine (3) and N-hydroxy-N′,N″-diphenyl guanidine (4)

Column: LiChrospher RP-select B (250×4.6 mm, 5 μm)

Column temperature: 20° C.

Mobile phase: 70% (v/v) 40 mM ammonium acetate, pH 5.2 30% (v/v) acetonitrile

Flow rate: 1.0 ml/min

Run time: 15 min.

Detection: Absorption measurement at 229 nm

Injection volume: 20 μl

Retention times:

| | |
|---|---|
| N-carboxymethoxy-N′,N″-diphenyl guanidine (3) | 5.2 min + 0.1 min |
| N-hydroxy-N′,N″-diphenyl guanidine (4) | 9.0 min + 0.2 min |

### Calibration and Recovery

For the calibration, N-hydroxy-N′,N″-diphenyl guanidine (4) was dissolved in eight concentrations of 0.1-500 μM, dissolved in assay buffer (30 mM MES, 1 μl copper(II) acetate, 2 mM sodium ascorbate, 5 mM potassium iodide, pH 6.0), and measured using the aforementioned HPLC method.

For determining the recovery, the same concentrations were produced in assay buffer (end volume=100 μl). In addition, N-carboxymethoxy-N′,N″-diphenyl guanidine (3) (0.5 mM) and 250 U/ml catalase were added, followed by 50 μl 10% TFA_{(aq)/}acetonitrile (2:3). The samples were shaken using the vortexer and frozen at −80° C. To measure the samples, they were thawed, shaken 5 minutes using the vortexer, and centrifuged for 5 minutes at 10000 rpm.

### CYP450 Assay: Incubation Conditions

A typical incubation bath of 500 μl (total volume) contained 0.3 mg protein (pork or human liver enzyme source), 0.1 mM (or 1 mM) test compound in 100 mM phosphate buffer (pH 6.0 or pH 7.4) and 1 mM NADH (or NADPH). The incubation was started after a 5-minute pre-incubation of the enzyme and test compound in buffer, adding NADH (or NADPH), and the product was shaken for 60 minutes or 180 minutes at 37° C. in the shaking water batch. The batches were stopped by adding the same volume of acetonitrile, shaken using the vortexer, and frozen at −80° C.

To analyze the samples, they were thawed, shaken 5 minutes using the vortexer, and the protein was separated by means of 5-minute centrifugation at 10000 rpm. The supernatant was used for the HPLC analysis.

HPLC Analysis for Separating O-carboxymethyl benzamidoxime (1), benzamidoxime (2) and benzamidine

Column: Synergi Max-RP 80 A (250×4.6 mm, 4 μm)

Column temperature: 20° C.

Mobile phase: 82.5% (v/v) 10 mM octyl sulfonate, pH 2.5 (H₃PO₄) 17.5% (v/v) acetonitrile

Flow rate: 1.0 ml/min

Run time: 35 min.

Detection: Absorption measurement at 229 nm

Injection volume: 20 μL

Retention times:

| | |
|---|---|
| O-carboxymethyl benzamidoxime (1) | 13.6 min + 0.1 min |
| Benzamidoxime (2) | 22.8 min + 0.3 min |
| Benzamidine | 26.0 min + 0.3 min |

HPLC Analysis for Separating N-carboxymethoxy-N′,N″-diphenyl guanidine (3), N-hydroxy-N′,N″-diphenyl guanidine (4) and N′,N″-diphenyl guanidine

Column: LiChrospher RP-select B (250×4.6 mm, 5 μm)

Column temperature: 20° C.

Mobile phase: 80% 20 mM ammonium acetate, pH 4.3 20% acetonitrile

Flow rate: 1.25 ml/min

Run time: 15 min.

Detection: Absorption measurement at 205 nm

Injection volume: 30 μL

Retention times:

| | |
|---|---|
| N,N′-diphenyl guanidine | 6.7 min + 0.2 min |
| N-carboxymethoxy-N′N″-diphenyl guanidine (3) | 7.8 min + 0.2 min |
| N-hydroxy-N′,N″-diphenyl guanidine (4) | 10.7 min + 0.3 min |

Hereinafter a table is provided of the pharmaceuticals to which the prodrug system according to the invention can preferably be applied:

| | Substance | | |
|---|---|---|---|
| Structure | name | CAS | Action/indication |
| 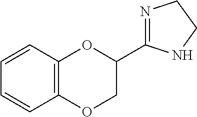 | idazoxan hydrochloride | 79944-58-4 | selective α2 adrenergic receptor antagonist and antagonist of the imidazoline receptor; initially tested as anti-depressant, but now being examined for schizophrenia |
| 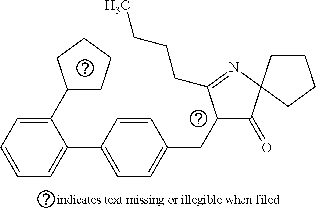 | irbesartan | 138402-11-6 | anpotensin II receptor antagonist, like most sartans against high blood pressure |
| 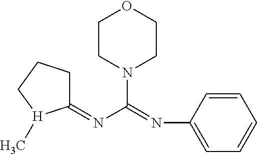 | linogliride | 75358-37-1 | against hyperglycemia |
| 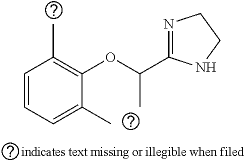 | lofexidine hydrochloride | 21498-08-8 | α2 adrenergic receptor antagonist, previously as a blood pressure reducing agent, today primarily used against heroin and opiate withdrawal symptoms |
| 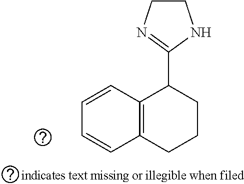 | tetra- hydrozoline hydrochloride | 522-48-5 | in eye drops and nasal sprays, alpha antagonist |
| 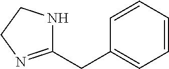 | tolazoline | 59-98-3 | non-selective, competitive α2 adrenergic receptor antagonist; has vessel-expanding effect, usually used in veterinary medicine as a wake-up agent |
| 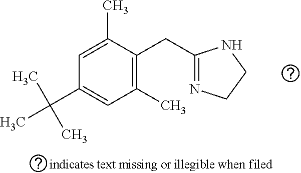 | xylo- metazoline hydro- chloride | 1218-35-5 | nasal spray against the cold, etc. |
| 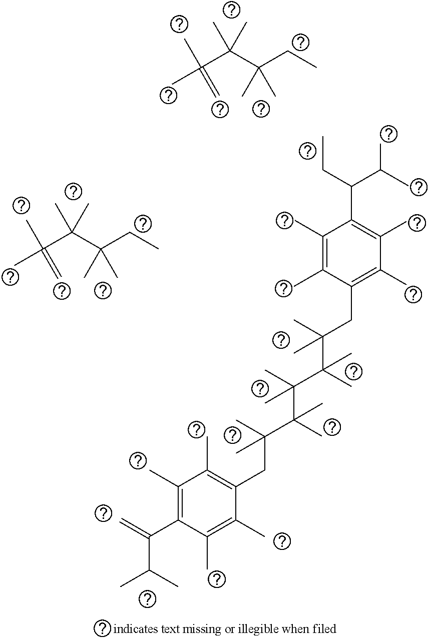 | pentamidine isethionate | 140-64-7 | anti-infectious antiprotozoal gent effective in trypanosomiasis, leishmaniasis and some fungal infections |
| 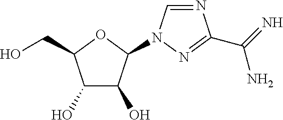 | taribavirin | 119567-79-2 | polymerase inhihitor against hepatitis C |
| 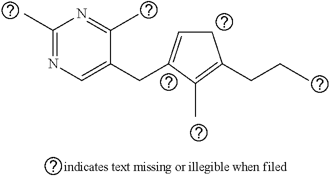 | thiamine (Vitamin B1) | 59-43-8 | Vitamin B1 deficiency |
| 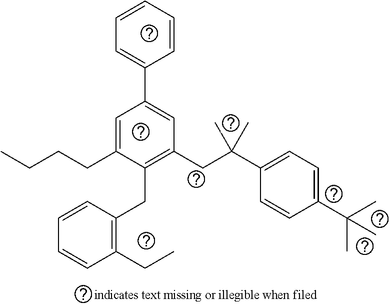 | bosentan | 147536-97-8 | endothelin receptor antagonist for treating pulmonary arterial hypertension |
| 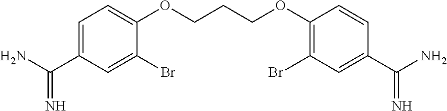 | dibromo- propamine isethionate | 496-00-4 | antiseptic, eye drops |
| 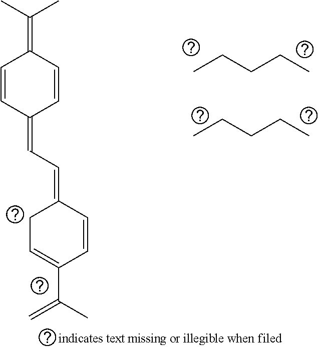 | hydroxy- stilbamidine isothionate | 495-99-8 | treating various fungal infections |
| 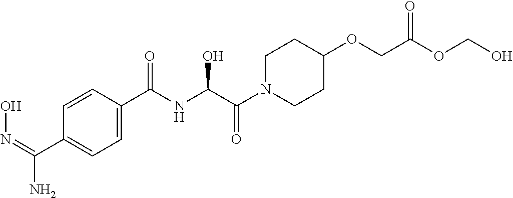 | sibrafiban | 172927-65-0 | GPIIb/IIIA inhibitor |
| 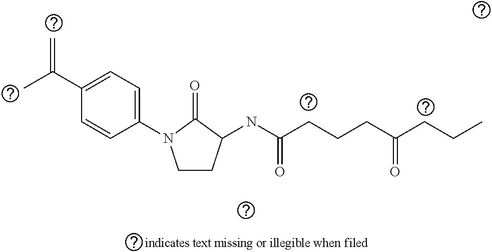 | orboliban | 163250-90-6 | antiplatelet drug |
| 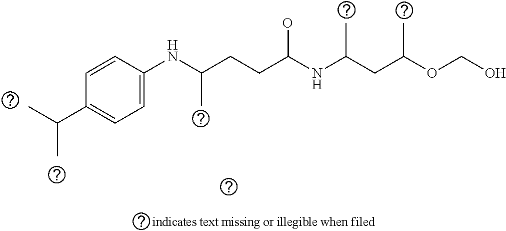 | xemilofthan | 149820-74-8 | glycoprotein IIb/IIa antagonist as coagulation inhibitor |
| 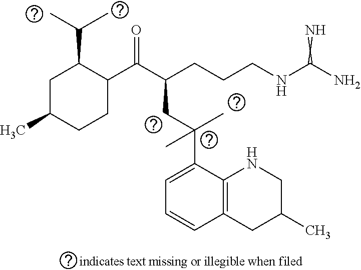 | argatrohan | 74863-84-6 141396-28-3 | anticoagulants |
| 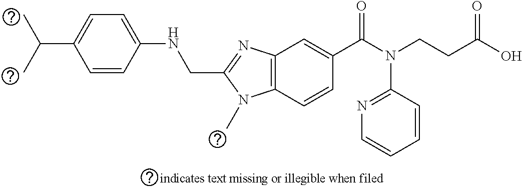 | dabigatran | 211941-51-1 | anticoagulants |
| 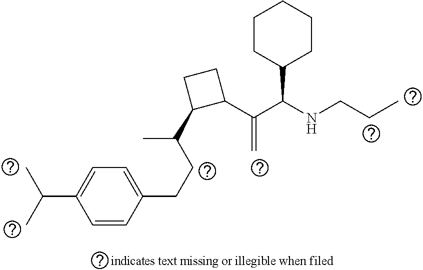 | ximelagatran/ melagatran | 159776-70-2 | thrombin inhibitor |
| 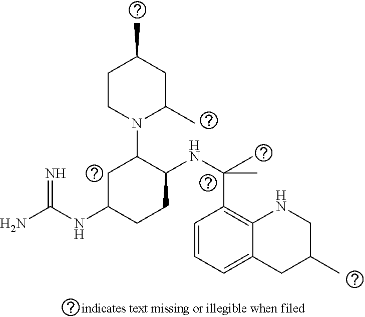 | 2-piperidine carboxylic acid | 74863-84-6 | thrombin inhibitor |
| 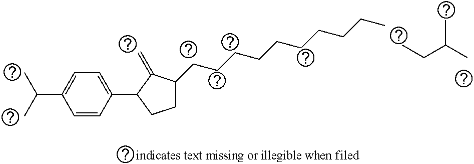 | orbofiban acetate | 165800-05-5 | glycoprotein IIb/IIIa receptor antagonist |
| 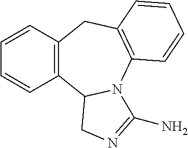 | epinastine (Relestat) | 127786-29-2 | antihistamine |
| 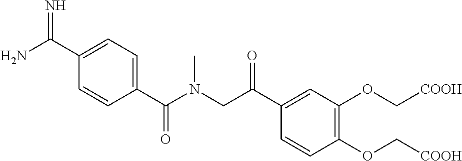 | RO 43-8857 | 1322224-71-6 | GPIIb/IIIa antagonist, anticoagulant |
| 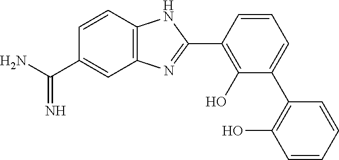 | | | FVIIa inhibitor, anticoagulant, preclinical |
| 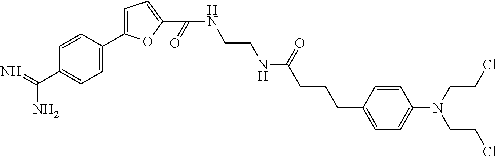 | AB (Chloram- bucil analogues) | 305-03-3 | leukemia, lymphomas, breast cancer, preclinical |
| 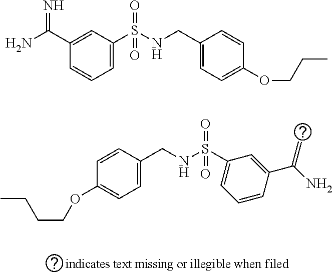 | AMG- 126737 | 224054-76-6 | tryptase inhibitor, preclinical |
| 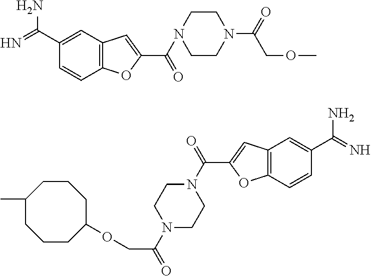 | AY-0068 | | tryptase inhibitor, preclinical |
| 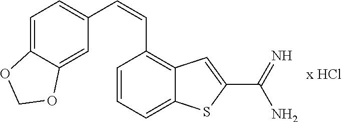 | B-623 | | urokinase inhibitor, malignant diseases, preclinical |
| 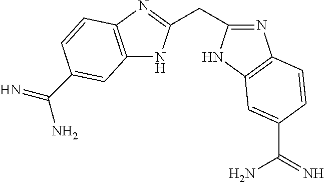 | BABIM | 74733-75-8 | tryptase inhibitor, allergies, asthma, preclinical |
| 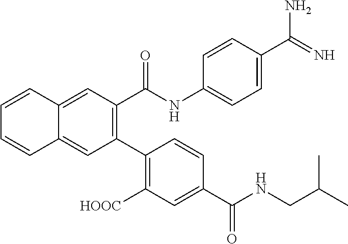 | | 1670-14-0 | factor VIIa inhibitor, anticoagulant, preclinical |
| 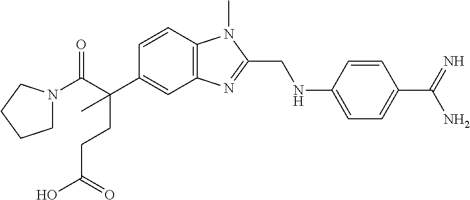 | BIBT-986 (Tanogitran) (Boehringer Ingelheim) | 637328-69-9 | dual FXa and thrombin inhibitors, anticoagulant |
| 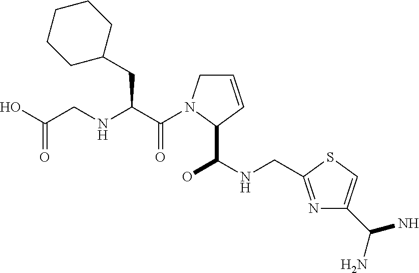 | BSF 411693 (Abbott) | | thrombin inhibitor, anticoagulant, preclinical |
| 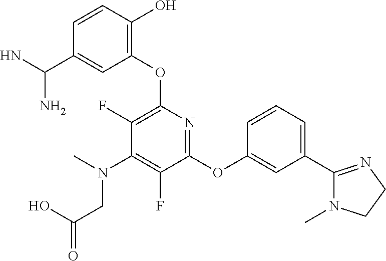 | CI-1031 (Biosciences) | 605-69-6 | FXa inhibitor, anticoagulant, Phase II |
| 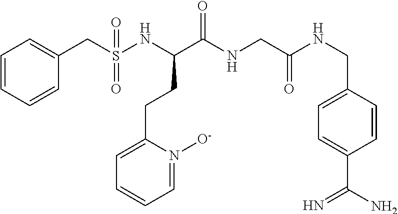 | CJ-1332 (Curacyte) | | FXa inhibitor, anticoagulant, preclinical |
| 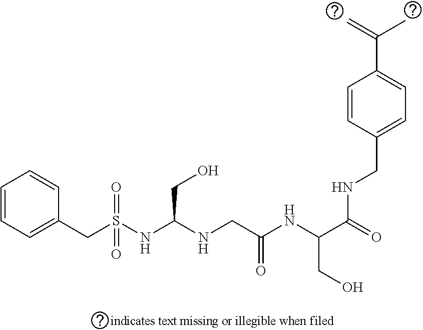 | CJ-463 (Curacyte) | | urokinase inhibitor, malignant diseases, preclinical |
| 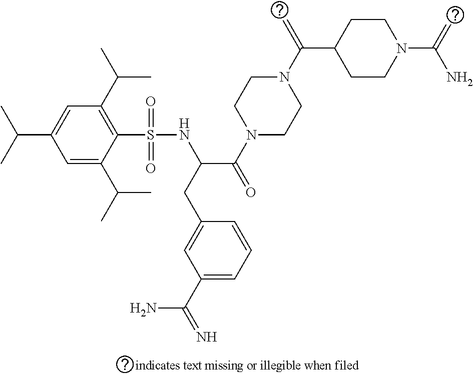 | CJ-672 (Curacyte) | | matriptase inhibitor, therapy of malignant diseases, preclinical |
| 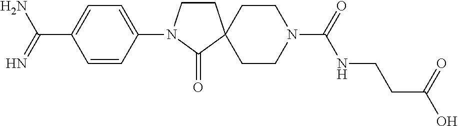 | CT50728 (Portolla Pharma- ceuticals) | | glycoprotein IIb/IIa antagonist as coagulation inhibitor |
| 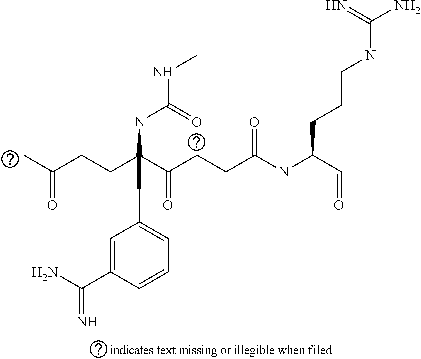 | CVS-3983 | | matriptase inhibitor, malignant diseases, preclinical |
| 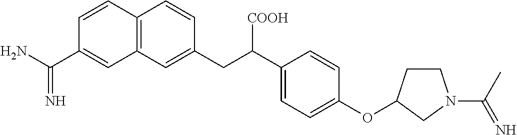 | DX-0065a | 155204-81-2 | FXa inhibitor, anticoagulant, Phase III |
| 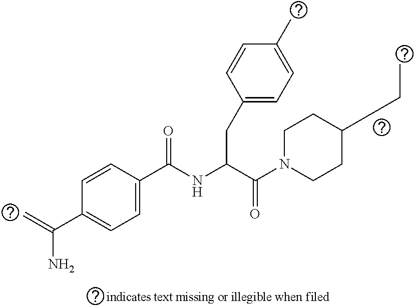 | Lamiliban (Roche) | 103577-45-3 | glycoprotein IIb/IIa agonist as coagulation inhibitor |
| 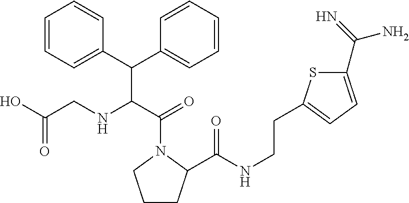 | LB-30870 (LG LifeSciences Ltd) | | thrombin inhihitor, anticoagulant, Phase I |
| 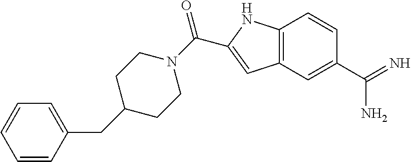 | LY-178550 (Lilly) | | thrombin inhibitor, anticoagulant, preclinical |
| 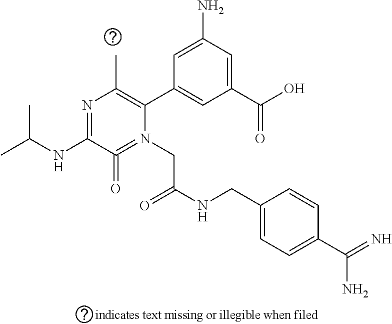 | PHA-927F and analogues | | factor Va inhibitor, anticoagulant |
| 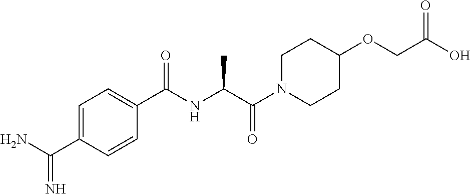 | RO-44-3888 (Roche) | | glycoprotein IIb/IIa antagonist as coagulation inhibitor |
| 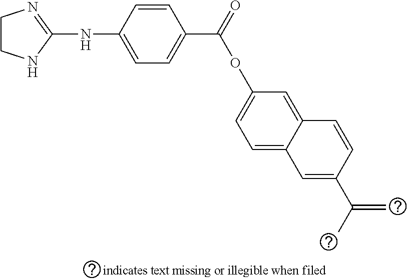 | sepimostat, FUT-187 (Torii) | 103926-64-3 | C3/C5 convertase inhibitor, complement activation, antiphlogistic, Phase II |
| 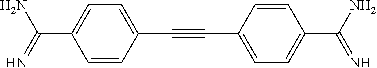 | stilbamidine (analogue diminazene, berenil) | 140-59-0 | trypanosomiasis |
| 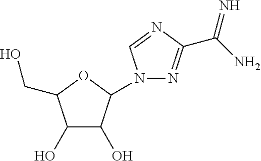 | viramidine (Ribapharm) | 119567-79-2 | static agent (hepatitis C), Phase III |
| 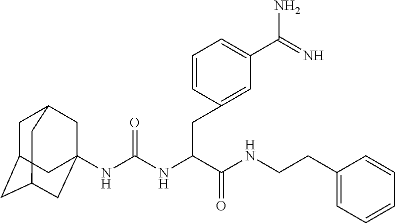 | WX-FX4 (Wilex) | | FXa inhibitor, anticoagulant, preclinical |
| 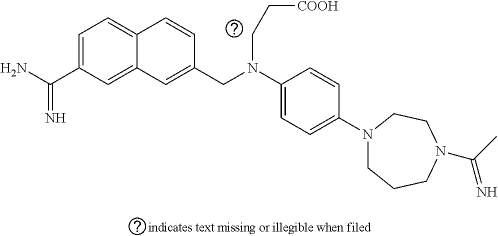 | YM-60828 (Yamanouchi Pharma- ceutical Co. Ltd.) | | FXa inhibitor, anticoagulant, preclinical |
| 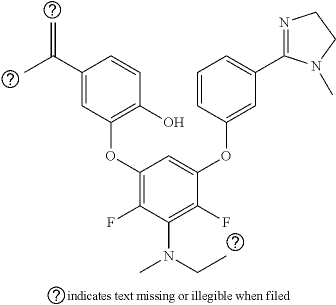 | ZK-707191 (Berlex Biosciences) | | FXa inhibitor, clinical testing |
| 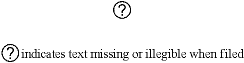 | NAPAP, SR25477 | 86845-59-2 | analgesic |
| 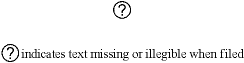 | BIIL 315 (Boehringer Ingelheim) | 204074-94-7 | inflammation inhibitor |
| 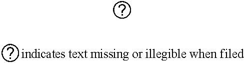 | BIIL 284/260 (Boehringer Ingelheim) | 204974-93-6 | inflammation inhihitor |
| 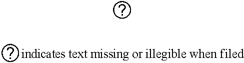 | tanogitran | 637328-09-9 | thrombin inhihitor |
| 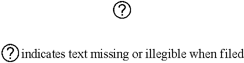 | moxiluhant | 146978-48-5 | antiasthmatic agent, antiinfective agent |
| 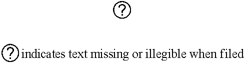 | stilbamidine | 122-06-5 | antiprotozoal agent, antiinfective agent. fungistatic agent |
| 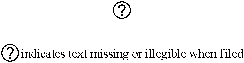 | panamidinc | 104-32-5 | antiprotozoal agent, antiinfective agent |
| 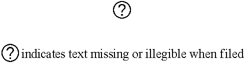 | fradafiban | 148396-36-5 | thrombin inhibitor |
| 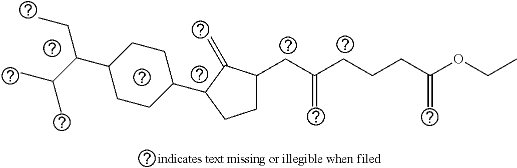 | orbofiban | 163250-90-6 | glycoprotein IIb/IIa antagonist as coagulation inhibitor |
| 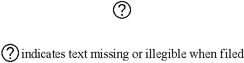 | roxifihan | 170902-47-3 | glycoprotein IIb/IIa antagonist as coagulation inhibitor |
| 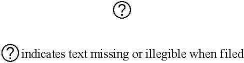 | lamifiban (Roche) | 144412-49-7 | thrombin inhibitor |
| 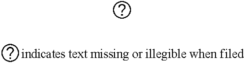 | furamidine | 73819-26-8 | antiprotozoal agent, antiinfective agent |
| 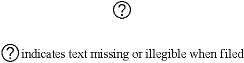 | PD0313052 | 861244-44-2 | thrombin inhibitor |
| 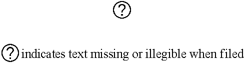 | PHA 927 F | 648943-12-8 | specific TF/VIIa inhibitor |
| 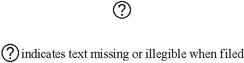 | PHA 798 | 508173-28-2 | specific TF/VIIa inhibitor |
| 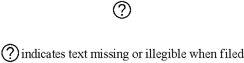 | stilbamidine | 122-06-5 | antiprotozoal agent, annfective agent |
| 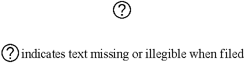 | fidexahan (ZK807834) | 183305-24-0 | anticoagulant |
| 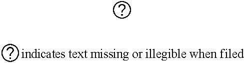 | otamixaban | 193153-04-7 | factor Xa inhibitor |
| 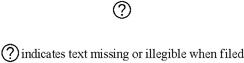 | thrombostop | 117091-16-4 | thrombin inhibitor |
| 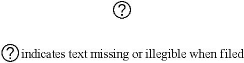 | amiloride hydrochloride | 2016-88-8 | diuretic |
| 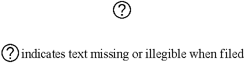 | anagrelide hydrochloride | 58579-51-4 | reduces thrombocytes |
| 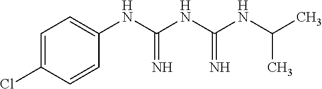 | proguanil | 537-21-3 | prophylactic anti-malaria agent (against plasmodium) |
| 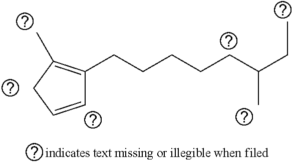 | cimetidine | 51481-61-9 | H2 receptor antagonist (against heartburn) |
| 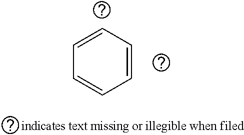 | clonidine hydrochloride | 4205-91-8 | direct α2 adrenergic agonist treatment of hypertension *treatment of drug withdrawal symptoms (alcohol, opioids etc) *No monotherapy for alcohol |
| 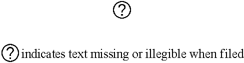 | guanoxan | 19694-60-1 | blood pressure reducing agent |
| 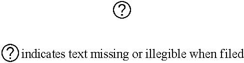 | peramivir | 229614-55-5 | neuramidase inhibitor (influenza), approved for emergencies with H1N1 infections, intravenous |
| 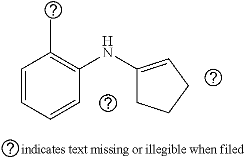 | romifidine | 65896-14-2 | α2 adrenergic receptor antagonist, veterinary medicine: sedative, anesthetic, analgesic for large animals like horses |
| 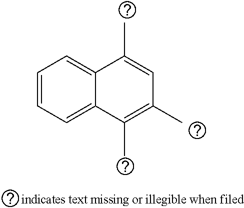 | tirapazamine | 27314-97-2 | experimental anti-tumor agent, releases small amounts of toxic radicals, chemical lead for other cancer drugs |
| 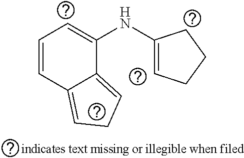 | tizanidine | 51322-75-9 | α2 adrenergic receptor antagonist muscle relaxant against spasms, cramps etc. |
| 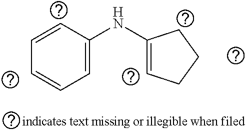 | tolonidine nitritc | 57524-15-9 | |
| 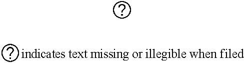 | metformin | 657-24-9 | diabetes mellitus type 2 |
| 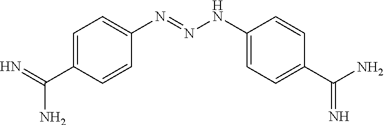 | diminazene | 536-71-0 | antiprotozoal agent, antiinfective agent |
| 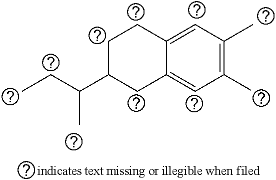 | debrisoquine | 1131-64-2 | antihypertension agent |
| 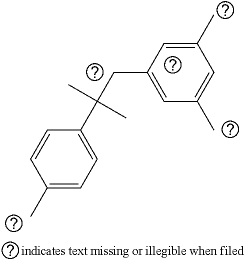 | sulfa- methazine | 57-68-1 | additive in animal feed |
| 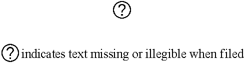 | eptifibatide | 188627-80-7 148031-34-9 | anticoagulant |
| 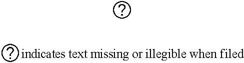 | famotidine | 76824-35-6 | Na+ H+ transport inhibitor |
| 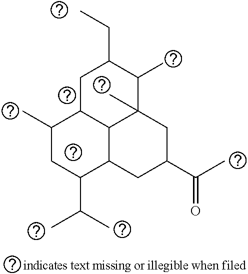 | zanamivir | 139110-80-8 | neuramidase inhibitor (influenza) |
| 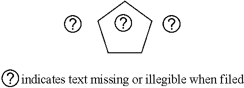 | Bayer pharma- ceutical | 25836-74-2 | |
| 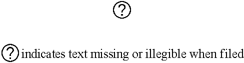 | streptomycin A | 57-92-1 | antibiotic |
| 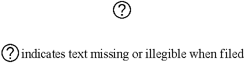 | nafamostat | 81525-10-2 | |
| 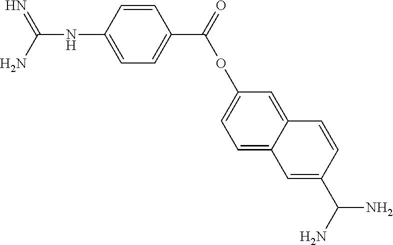 | nafamostat, FUT-175 | 81525-10-2 | C3/C5 convertase inhibitor, complement, activation, antiphlogistic, Phase II |
| 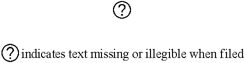 | inogatran | 155415-08-0 | |
| 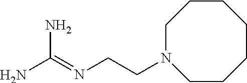 | guanethidine (Thilodigon) | 645-43-2 | hypertension agent, glaucoma |
| 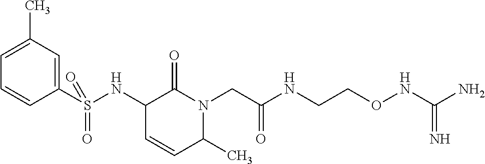 | 3DP-10017 | 226566-43-4 | dual FXa and thrombin inhibitor, clinical development |
| 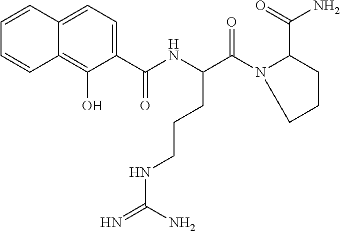 | APC-366 | 178925-65-0 | tryptase inhibitor, allergics, asthma, Phase II |
| 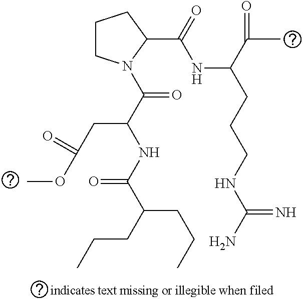 | CVS-1123 | | thrombin inhibitor, clinical testing |
| 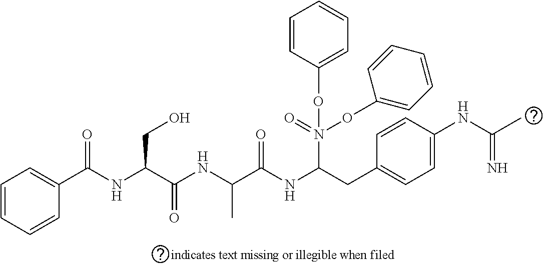 | diphenyl phosphonate derivatives | 5945-33-5 | urokinase inhibitor; malignant diseases. preclinical |
| 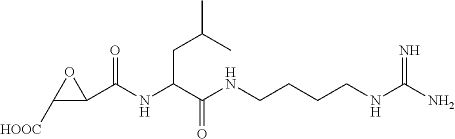 | E-64 | 66701-25-5 | Cathepsin L inhibitor, malignant diseases, preclinical |
| 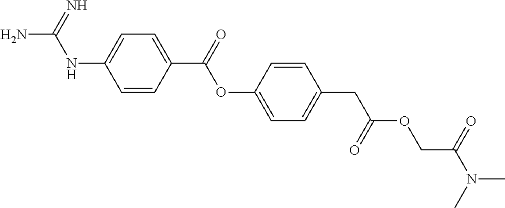 | FOY-305 | 59721-28-7 | broad spectrum scrip protease inhibitors (pancreatitis, anticoagulation), clin. development |
| 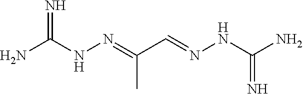 | MBGB | | lymhomas. leukemia, clinic |
| 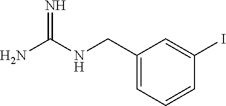 | MIBG | 103346-16-3 | neuroblastoma, clinic |
| 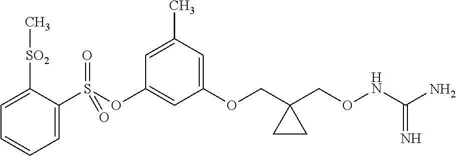 | RMJ-422521 | | dual FXa and thrombin inhibitor, clinical development |
| 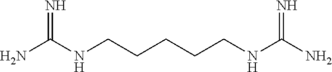 | Synthalin | 301-15-5 | antidiabetic agent |
| 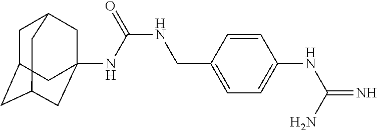 | WX-293 | | urokinase inhibitor, maligmant diseases, preclinical |
| 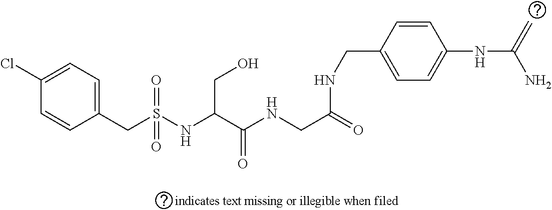 | WX-340 | | amyotrophic lateral sclerosis, malignomas, Phase I |
| 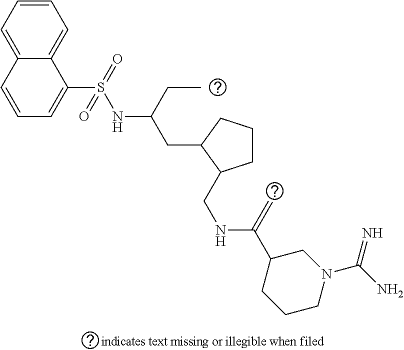 | BMS-189090 | | thrombin inhibitor, clinical testing |
| 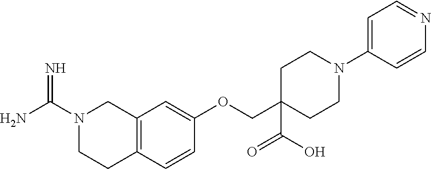 | JTV-803 (Japan Tobacco) | | FXa inhibitor, anticoagulant, Phase II |
| 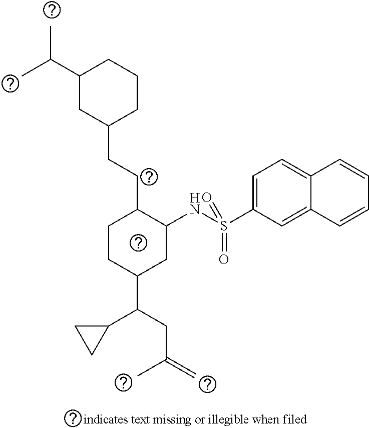 | napsagatran | 159668-20-9 | thrombin inhibitor, anticoagulant, clinical development |
| 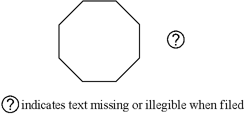 | Abapressin, azocine, Dopom | 55-65-2 | hypertension agent |
| 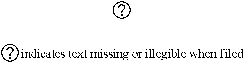 | TAN 1057A | 128126-44-3 | antibiotic |
| 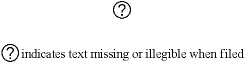 | Hydikal, MK 870 | 2609-46-3 | cardiac rhythm disorder |
| 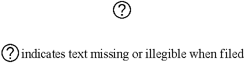 | Phenformin, Retardo | 114-86-3 | diabetes mellitus II |
| 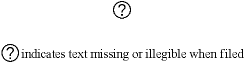 | netropsin, Sinanomycin | 1438-30-8 | antibiotic |
| 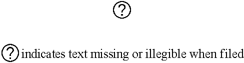 | BIIB, sabiporide | 261505-80-0 | Na + T+ transport hibitor |
| 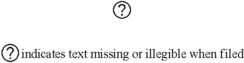 | deoxys- pergualin | 89149-10-0 | immunosuppressant |
| 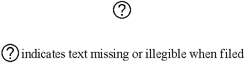 | | | |
| 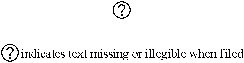 | BMS 262084 | 253174-92-4 | tryptase inhibitor |
| 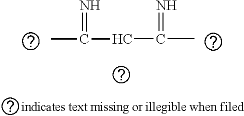 | Apophage, Siamformet | 1115-70-4 | diabetes mellitus II |
| 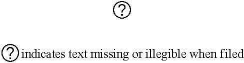 | Pebac, L-prolinamide | 71142-71-7 | thrombin inhihitor |
| 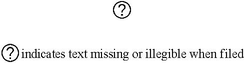 | MERGETPA | 77102-284 | thrombin inhibitor |
| 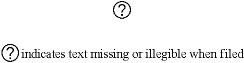 | BCX 1812, peramivir | 330600-85-6 | neuramidase inhibitor (influenza) |
| 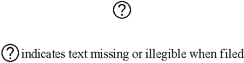 | apogastine, famogast | 76824-35-6 | proton pump inhibitor |
| 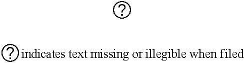 | | 65113-67-9 | |
| 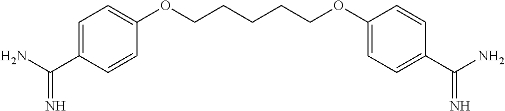 | pentamidine | 100-33-4 | antiprotozoal agent, antiinrective agent |

1. A prodrug, comprising a partial structure having the general formula (I) or (II) 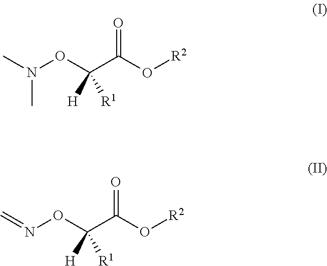
where R¹ and R² are hydrogen, alky radicals or aryl radicals.

2. The prodrug according to claim 1, characterized in that the partial structure which the prodrug comprises is part of a hydroxylamine, an N-oxide, a nitron, a diazeniumdiolate (NONOat) or a similar N—O-containing nitric oxide donor, a hydroxamic acid, a hydroxyurea, an oxime, an amidoxime (N-hydroxyamidine), an N-hydroxyamidinohydrazone or an N-hydroxyguanidine.

3. A prodrug according to any one of the preceding claims, characterized in that the prodrug is metabolized into a pharmaceutical, which is a pharmaceutical for treating diseases associated with nitric oxide deficiency.

4. A prodrug according to any one of the preceding claims, characterized in that the prodrug or the corresponding pharmaceutical is selected from the group consisting of protease inhibitors, DNA- and RNA-intercalating compounds, inhibitors of viral enzymes, and N-methyl-D-aspartate receptor antagonists.

5. A prodrug according to any one of claims 1 to 4, characterized in that the partial structure has the general formula IIa or IIb

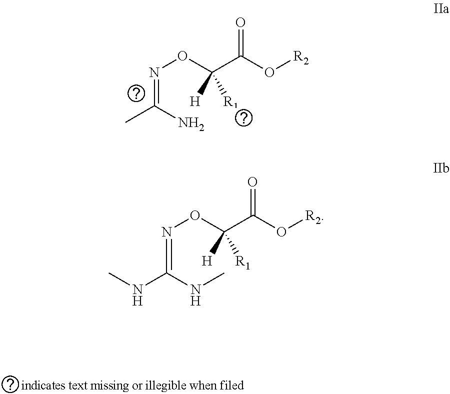

6. The prodrug according to claim 5, characterized in that the prodrug is a prodrug of a pharmaceutical, wherein the partial structure of the general formula IIa, after metabolization, comprises a structure having the formula 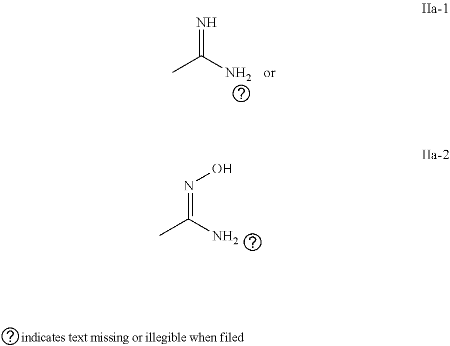
and the partial structure of the general formula IIb, after metabolization, comprises a structure having the formula

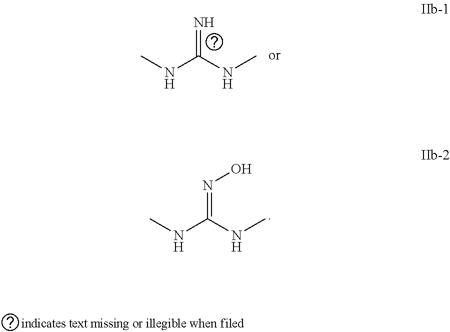

7. Use of a partial structure forming the general formula (I) or (II) 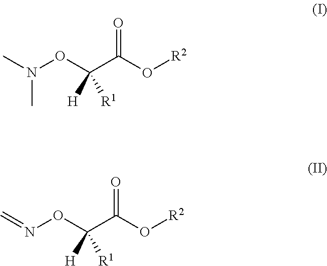
as part of the overall structure of a prodrug which is prodrug of a pharmaceutical, where R¹ and R² are hydrogen, alkyl radicals or aryl radicals.

8. Use according to claim 7, wherein the partial structure has the general formula (II), which is part of a higher-level partial structure IIa or IIb 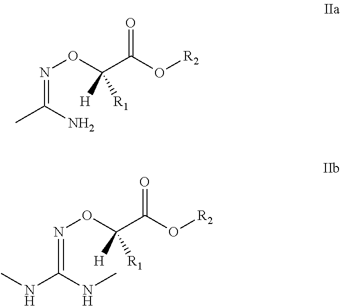
in the place of an amidine or guanidine group of a pharmaceutical to improve solubility, oral bioavailability, blood-brain barrier crossing, the flavor and/or the physical-chemical stability.

9. Use according to claim 7 or 8, wherein the prodrug is a prodrug of a pharmaceutical which has the same structure as the prodrug, except that instead of the higher-level partial structure IIa it comprises one of the partial structures IIa-1 or IIa-2 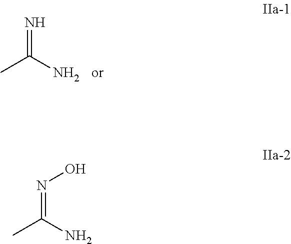
or instead of the higher-level partial structure IIb it comprises one of the partial structures IIb-1 or IIb-2

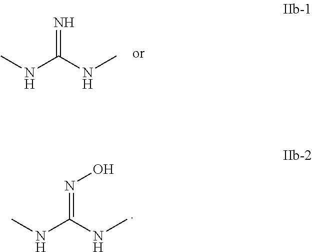

10. Use according to claim 8 or 9 for activating the pharmaceutical by means of peptidylglycine α-amidating monooxygenase (PAM).

11. Use according to any one of claims 7 to 10, characterized in that the partial structure is part of a hydroxylamine, an N-oxide, a nitron, a diazeniumdiolate (NONOat) or a similar N—O-containing nitric oxide donor, a hydroxamic acid, a hydroxyurea, an oxime, an amidoxime (N-hydroxyamidine), an N-hydroxyamidinohydrazone or an N-hydroxyguanidine.

12. A method for introducing a pharmaceutical comprising a free amidine or guanidine function into the PAM activation path, comprising the production of a prodrug of the pharmaceutical according to any one of claims 1 to 6.

13. A method for treating a patient, comprising the administration of a prodrug according to any one of claims 1 to 6 to the patient.

14. Use of a prodrug according to any one of the claims 1 to 6 for producing a pharmaceutical.

15. Use according to any one of claims 7 to 11 and claim 13, or the method according to claim 14, wherein the use or the method is a use or a method for treating diseases associated with nitric oxide deficiency.

16. Use according to any one of claims 7 to 11 and 15, characterized in that the pharmaceutical or the prodrug is selected from the group consisting of protease inhibitors, DNA- and RNA-intercalating compounds, inhibitors of viral enzymes, and N-methyl-D-aspartate receptor antagonists.

17. Use according to any one of claims 7 to 11 and 15 to 17, wherein the use is a use for the prophylaxis and/or treatment of visceral and/or cutaneous leishmaniasis, trypanosomiasis, phase 2 of trypanosomiasis or pneumonia caused by Pneumocystis carinii, for inhibiting the growth of malignant tumors, for inhibiting blood coagulation, for lowering blood pressure, for neuroprotection, or for combating viral infections, including influenza and HIV infections.

18. A method for producing an N-alkoxy guanidine of the formula 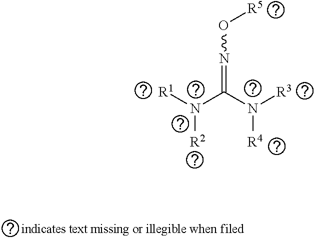
comprising the reaction of a carbodiimide of the formula R₆—N═C═N—R₇ with an aminooxy compound of the formula H₂N—O—R₈ or a salt thereof, wherein when a salt of the aminooxy compound is used, the reaction is carried out in the presence of a base, where R1, R2, R3 and R4, R6 and R7 independently of each other are selected from the group consisting of H, optionally substituted alkyl, optionally substituted aryl, optionally substituted heteroaryl, optionally substituted aryloxycarbonyl, optionally substituted aminoacyl, optionally substituted alkoxycarbonyl, optionally substituted alkoxycarbonylalkoxy, optionally substituted heteroalkyl, optionally substituted alkylcycloalkyl, optionally substituted heteroalkyl cycloalkyl, optionally substituted aralkyl, and optionally substituted cycloalkyl,
and R5 is selected from the group consisting of alkoxycarbonyl, (alkoxycarbonyl)alkoxy and carboxyalkoxy.

19. The method according to claim 18, wherein the reaction is carried out in the presence of a base, which is preferably selected from the group consisting of diisopropylamine and triethylamine.
